(19) United States
(12) Patent Application Publication
(10) Pub. No.: US 2004/0045546 A1
(43) Pub. Date: Mar. 11, 2004

(54) Pharmaceutical delivery system for oral inhalation through nebulization consisting of inert substrate impregnated with substance (S) to be solubilized or suspended prior to use

(75) Inventors: Jane Hirsh, Wellesley, MA (US); Whe-Yong Lo, Canton, MA (US)

Correspondence Address: PATREA L. PABST, HOLLAND & KNIGHT LLP, SUITE 2000, ONE ATLANTIC CENTER, 1201 WEST PEACHTREE STREET, N.E., ATLANTA, GA, 30309-3400, US

(73) Assignee: PEIRCE MANAGEMENT, LLC

(21) Appl. No.: 10/242,803

(22) Filed: Sep. 5, 2002

(51) Int. Cl. A61M 11/00; A61M 15/00

(52) U.S. Cl. 128/200.14; 128/200.16

(57) ABSTRACT

A pharmaceutical delivery system for oral inhalation is disclosed through nebulization consisting of an inert supporting material impregnated with or deposited with pharmaceutically active ingredient which must be solubilized or suspended in a pharmaceutical solvent to form a solution or suspension prior to administration. Each pharmaceutical delivery unit dosage form comprises one or more therapeutically effective and safe amounts of pharmaceutically active ingredient uniformly impregnated in or deposited on a supporting material which is a natural or synthetic polymer, woven or non-woven fabrics, inert paper, inorganic materials such as foil and combination thereof in a single or multi-layer lamination in a form of a sheet or strip or film or membrane or sponge-like or cup or well. The dosage form of this invention is to be administered to a patient through oral or nasal inhalation using a nebulizer after reconstitution with a reconstituting solvent.

### SPECIFICATION

1. Field of the Invention

The invention relates to the delivery of pharmaceutical active substance to the respiratory system through a liquid for oral or nasal inhalation by a nebulizer. This invention further relates to compositions, method of preparation, and method of use for a pharmaceutical dosage unit comprising one or more therapeutically effective amounts of pharmaceutical active ingredients for oral inhalation.

2. Background of the Invention

Remington, The Science and Practice of Pharmacy 20^{th} edition, Chapter 50 teaches that “Inhalation therapy has been used for many years, and there has been a resurgence of interest in delivery of drugs by this route of administration. The number of new drug entities has increased over the last 5 to 10 years, as well as many of the existing drugs delivered to the body via the respiratory system. This type of therapy also has been applied to delivery of drugs through the nasal mucosa as well as the oral cavity for buccal absorption.

Oral inhalations are drugs or solutions or suspensions of drugs administered by the oral respiratory route. The drugs may be administered for their local action on the bronchial tree or for their systemic effects through absorption from the lungs. For the inhaled drug substance or solution to reach the bronchial tree, the inhaled particles must be just a few micrometers (0.5 to 10, preferably 0.5 to 5 micrometers) in size. Inhalation therapy is used primarily to administer drugs directly to the respiratory system mostly for treatment of bronchospasms, mucosal enema, pulmonary infections and the like. Delivery of therapeutic agents directly to affected respiratory tracts has several advantages. The drug reaches the target tissue without first entering the systemic circulation where the drug molecules are subjected to dilution, metabolism, distribution and excretion. A high local concentration of drug can be reached in the lungs while the systemic concentration is kept below that likely to cause adverse side effects. Inhalation therapy is also now being used for drugs to be delivered to the bloodstream and finally to the desired site of action. Oral inhalation dosage forms for protein, steroids, cardiac agents, immunizing agents, etc, are either under development or currently marketed.

Oral inhalations may be administered through three types of devices: (1) a pressure packaged inhalation aerosol such as a metered dose aerosol (MDI), (2) a dry powder inhaler for dry powder inhalation (DPI), and (3) a nebulizer or an atomizer, most commonly either a compressor driven jet nebulizer or ultrasonic nebulizer. The jet nebulizer is driven by compressed air creating turbulence and hence mist, while the ultrasonic nebulizer has a small plate that vibrates at ultrasonic frequency to create mist.

With the MDI, the drug is formulated and pre-packaged in a pressurized container with a metered dose valve. The unit is placed within an oral adapter (mouth piece), and when the unit is actuated, an exact amount of drug is expelled in the proper particle size distribution to achieve maximum absorption of drug into the lungs while the patient inhales. With the DPI, a therapeutically effective amount of micronized drug powder for one dose is delivered from a pre-packaged capsule or from a drug holding chamber of the DPI device. The micronized drug is formulated and prepackaged in a gelatin or cellulose capsule or in a drug holding chamber of a DPI device. The release and delivery of drug particles from the capsule or from the drug holding chamber is activated through respiratory inhalation by the patient. A nebulizer is operated by instilling with a calibrated dropper or pouring a defined liquid amount of a drug to be inhaled in a solution or suspension into the chamber or reservoir of a nebulizing device. Upon the application of atomized air or sonication force, the drug solution forms a fine mist, which is inhaled by the patient through a mouthpiece, or a mask, or as instructed by the medical staff.

The metered-dose inhalation aerosol dosage form, although popular, generally is considered one of the most complicated drug-systems for a patient. Administration of the pressurized aerosols requires (1) patient's coordination between actuating the aerosol and inspiration and (2) requires breath-holding for seconds after inspiration of the aerosol. On many occasions, patients can not differentiate between an empty or loaded MDI device and, as a result, inhalation is made without delivery of medicament. Similarly, administration of dry powder inhalations requires activation of the device by sucking from inspiration, followed by similar breath-holding. Failure of the patient to operate and inhale correctly may alter significantly the deposition of the drug into the appropriate portion of the airways. Therefore, for children who are too young or for patients who are too weak to effectively operate and inhale the pressurized aerosols and dry powder inhalers and for patients whose airways are so irritable that they will cough out medications inhaled from pressurized aerosols or dry powder inhalers, nebulizers that generate a fine mist or droplets of medicament and which are inhaled through either a mouthpiece or a mask as the patient breathes normally and does not require inhalation technique are the inhalation devices of choice.

Solutions or suspensions of pharmaceutically active ingredients in buffered saline and similar vehicles are commonly employed to generate an aerosol mist in a nebulizer. The inhalation solution or suspension may be formulated and prepared for administration without further dilution or may be prepared as a concentrate and further diluted to a desired concentration prior to nebulization. In general, 1 to 10 mL of the drug formulation are applied for nebulization with the inhalation process lasting from 5 to 30 minutes of several minutes. A typical nebulizer contains a reservoir, which holds the medicament solution and an atomizing unit, which may be compressor-driven or ultrasonically-driven. Application of compressed air or ultrasonic force to the drug reservoir produces a fine mist of drug solution with particle size range between 0.5 to 5 microns. The larger, heavier droplets of the mist do not exit the apparatus but fall back into the reservoir of medicated liquid. The lighter particles do escape with the air stream and are inhaled by the patient through the mouthpiece or a mask. Design and function of nebulizers are well known in the art and are readily commercially available from the market.

In general, medicaments to be used with a nebulizer are prepared in an aqueous solution for water soluble medicaments and are prepared in an aqueous suspension for water-insoluble medicaments. Examples of marketed solutions for oral inhalation products include water soluble drugs such as albuterol sulfate, acetylcysteine, bitolterol mesylate, cromolyn sodium, metaproterenol sulfate, epinephrine hydrochloride, levalbuterol hydrochloride and ipratropium bromide. Most of the inhalation solutions are formulated with sodium chloride as an isotonicity-adjusting agent. The solutions frequently contain disodium EDTA, citric acid buffer and an anti-microbial preservative agent for a multi-dose package. Unit of use solutions and suspensions are commercially available. For these Unit dose packages, an anti-microbial preservative agent is usually not required. However, the product is required to be manufactured under aseptic conditions.

Because of difficulties in stabilizing their particles in aqueous media, water insoluble or poorly water-soluble medicaments such as corticosteroids have usually been formulated as suspensions of micronized drug powder in chlorofluorocarbon or with chlorofluorocarbon-free propellants and delivered by metered-dose inhaler or have been formulated as a dry powder inhaler. Currently only one corticosteroids suspension for oral inhalation via a nebulizer is marketed—Pulmicort Respules by Astra Pharm. The suspension for Oral Inhalation contains budesonide (micronized) and the inactive ingredients disodium edetate, sodium chloride, sodium citrate, citric acid, polysorbate 80 and water for injection. The product is available in 2 mL sterile unit dose plastic containers and can be used directly without further dilution via a jet nebulizer connected to an air compressor. The suspension requires that it be manufactured under aseptic condition and packaged in unit dose containers that are then wrapped in aluminum foil to protect the product from light.

The Respules are packaged five doses per aluminum Pouch which when opened has a two weeks shelf life when protected from light. This short shelf life becomes expensive if the patient to discard the unused units.

There are a number of problems that arise during the formulation of aqueous nebulization solutions: (1) For medicaments that are chemically unstable in an aqueous medium, the aqueous dosage forms suffer from having a short shelf life or expiration date. Various methods have been tried to overcome the stability problems which have included (a) addition of a stabilizing agent, such as an antioxidant to the formulation, (b) manufacturing and packaging of the product under inert gas conditions, and (c) storing the product at a reduced controlled temperature ranging from refrigeration, to room temperature. Furthermore (d) packaging systems that are impervious to light, air or moisture have been tried to overcome the instability problem of the product. However, all the above approaches have drawbacks of either requiring addition of chemicals (e.g. an antioxidant or other stabilizers) to the formulation or requiring manufacturing under inert gas conditions and/or storing at a reduced temperatures that are all costly. The addition of an antioxidant or other stabilizing agent to the product formulation may potentially cause side effects including bronchospasms. See (Pharmacotherapy, A Pathophysiologic Approach, 4^{th} Ed., Ch. 27, pg 498, Publisher, Appletin and Lange 1999; Mathison D A, Stevenson D D, Simon R A, Precipitating factors in asthma: Aspirin, sulfites, and other drugs and chemicals. Chest 1985; 87(suppl):50-54; Bush R K, Taylor S L, Busse W. A critical evaluation of clinical trials in reactions to sulfites. J Allergy Clin Immunol 1986;78:191-202.). Manufacturing under inert gas conditions and stored product at controlled reduced temperatures are costly. (2) These aqueous solutions and suspensions are often supplied in a multi-dose container to be dispensed by the patient or medical staff using a dropper. Because the container is opened and closed multiple times, an anti-microbial preservative is added to preserve the product from microorganism contaminations where an antimicrobial preservative is added to preserve the product from microorganism contaminations. The addition of a preservative often has drawbacks that patients might develop hypersensitivity reactions to the preservative. Furthermore, the addition of a preservative may not always resolve the microbial contamination problem associated with multi dose containers. The patient may inadvertently overburden the preservative system during multiple re-entry into the container. Additionally, during the manufacturing process there is always a potential of degradation of the preservative system that can ultimately result in a product recall and consequently results in product failure. (3) Dispensing of a precise dosage amount of medicament from the multi-dose container may not always be achieved by the use of a calibrated dropper to be read at the meniscus at the time of administration. (4) To overcome the hypersensitivity problem from preservatives and to overcome the problem of a potentially inaccurate dosage dispensed from the multi-dose container, sterile unit dose packages containing an inhalation solution have been introduced to the market. Although these unit dose preparations do not require an anti-microbial preservative, the unit dose preparations are required to be manufactured under costly sterile conditions and there is a concern for the maintenance of the sterility of the package during packaging, shipping, storage and use. Generally these unit doses are plastic containers manufactured by an extrusion process that forms, fills and seals the dosage concurrently. This packaging process is subject to pin holding or wicking, which can result in an incomplete seal of the plastic over time. (5) For water-insoluble medicaments that are prepared in aqueous suspensions there is a probability that the drug particles will agglomerate into larger agglomerated particles over time and result in instability of the product. (6) In cold climates, liquid dosage forms may encounter alternating freezing and thawing conditions during transport, which can potentially cause physical instability such as precipitation. (7) For products that are light sensitive and/or not compatible with a plastic container, special Type II amber glass containers must be used. These glass containers must be handled with care and must be sterilized by either steam or gamma irradiation. Additionally the cap closure as well as the dropper system must be made sterile by appropriate methods. All components of the packaging system must be periodically monitored for bio-burden. These procedures which require microbiological facilities are costly but necessary.

Furthermore (8) handling of glass containers filled on high speed packaging lines can result in breakage which must be carefully monitored for resultant fragments. In addition, glass is inconvenient and costly to handle, ship, distribute and store. If the product dating expires before being dispensed to a patient it must be returned to the manufacturer where it will then be transported to special facilities to be destroyed by incineration; (9) Some aqueous unit dose preparations are packaged in aluminum foil pouches. Once the pouch is opened, the unit doses must be used within two weeks. Anything unused after two weeks must be discarded. This can be an undue expense to the patient. It is inconvenient and costly to handle, ship, distribute, store, and dispose of liquid products due to weight and space requirements.

To overcome some of the aforementioned problems associated with aqueous preparations for oral inhalation, a number of solutions have been revealed:

The invention described in U.S. Pat. No. 6,161,536 (M. Redman) relates to a dosage form for providing a precise dosage of water-sensitive medicament for administration as a nebulized aqueous aerosol. The patent requires medicament to be suspended in a solid state open matrix containing a water-soluble or water-dispersible carrier material which is formed by proteins such as gelatin, or polysaccharides such as alginate, or other carriers such as acacia. Additionally, the matrix may incorporate coloring agents, flavoring agents, preservatives, and the like. The solid state matrices are prepared by lyophilizing solvent from a composition comprising the medicament and a solution of the carrier material in a solvent. Although the solvent is primarily water, it may also contain a co-solvent such as 1-butanol when necessary to improve the solubility. To achieve the properties of the water-soluble or water-dispersible matrix of medicament and carrier, lyophilization is necessary. The difference in the present invention is that the inert supporting material is not water-dispersible or water-soluble. Only the medicament and the formulation excipients, if added, in the present invention are dispersible or soluble in water or non-aqueous solvents. Ideally, nebulization solutions should be formulated with a minimum of excipients and in the smallest amount as possible to minimize the possibility of unwanted or adverse reactions. The Redman patent requires that extraneous excipients such as gelatin, alginate and acacia, etc. be added to provide the open matrix network as well as bulk and to form the final lyophilized product. The delivery system of our present invention is an inert supporting material on which is deposited or in which is impregnated a therapeutically effective amount of at least one medicament and formulation excipients. The inert supporting material is neither dispersible nor dissolvable in the reconstituted solution and the major formulation excipients are of small molecules, such as sodium chloride, dextrose, lactose, pH adjusting agent, etc., instead of the macromolecules such as gelatin, alginate and acacia as mentioned in the Redman patent. These macromolecules might be unnecessary and unwanted as they become inhaled into the respiratory tract. Additionally the process of lyophilization which is an expensive processing step is not required in the present invention.

WO 99/44594 to S. Sonoke discloses a drug delivery system in which water-insoluble drugs are prepared as lipid-water emulsions, freeze-dried, and dispersed in water for nebulization. Like a suspension, emulsions have a general problem of becoming physically unstable. The emulsion breaks with phase separation to a state that the medicament particles are no longer dispersed evenly within the formulation. Emulsions have all the same stability concerns that are present with true solutions. An oil and a sufficient amount of surfactants are needed for the formulation of an emulsion and hence unnecessary excipients in a pharmacological sense are introduced into the composition to become inhaled into the respiratory tract. Additionally, Sonoke provides optionally for lyophilization of the emulsion; hence a matrix forming material has been incorporated. Again, lyophilization is required as an additional processing step that is not required in our present invention. Like other lyophilized products, the preparation of free-dried emulsion may be expensive.

U.S. Pat. No. 5,192,528 (R. Radhakrishnan) discloses water-insoluble or poorly water-soluble corticosteroids as liposomal formulations which are delivered to the patient by inhalation. The carrier consists of an aqueous suspension of sized liposomes containing the drug. This liposome-entrapped drug form is then aerosolized, using a nebulizer, to deliver the drug to the lungs. The present invention differs from Radhakrishnan's invention in that the pharmaceutically active compounds are not presented in the form of a solution or suspension initially, but reside on the inert supporting material in the dry state and are reconstituted to a solution or suspension only at the time of administration to a patient. Unlike the present invention, where the supporting material does not become part of the nebulizing solution or suspension, the liposomes themselves become part of the nebulizing suspension.

The most common cited problems that may arise with liposomes are reported as manufacturing process issues. Remington, the Science and Practice of Pharmacy 20^{th} Ed. Ch. 47, pg 919, A. Gennaro, Pub. Lippincott presents a brief overview of the concerns and issues that must be considered in formulations when employing liposomes. There are as yet no commercially marketed aerosolized liposomal preparations for nebulization available for pulmonary delivery of corticosteroids and other drugs. Furthermore there may be disadvantages to the patient in inhaling liposomes in terms of toxicity.

U.S. Pat. No. 6,241,969 to Z. Saidi discloses an aqueous formulation containing corticosteroids compounds as active agents for the treatment of ailments and diseases of the respiratory tract. The corticosteroids compounds are present in a dissolved state in the aqueous-based solution for ready delivery. The composition may contain high-HLB surfactant, tonicity-adjusting agents, buffer, co-solvents, and preservatives. The diluted ready-for-delivery formulation may be sterilized by passing them through a 0.22 micron sterile filter and may be filled into unit dose containers. The present invention differs from the Saidi invention in that our pharmaceutically active compounds are not to be presented in a form of solution or suspension initially, but reside on the inert supporting material in the dry state and are reconstituted to a solution or a suspension only at the time of administration to a patient.

These embodiments, as revealed in U.S. Pat. No. 5,192,528 and U.S. Pat. No. 6,241,969, although providing a ready-for-use aqueous formulation for nebulization, present the same disadvantages as other aqueous formulations described earlier.

WO 01/47491 discloses a formulation design of topically acting corticosteroids and other water-insoluble drugs for nebulizer inhalation in aqueous vehicles. In this invention, a water-insoluble drug is dissolved in a non-aqueous solvent at a sufficiently high concentration. A treatment dose in a measured small volume of the non-aqueous concentrate solutions can be mixed, immediately prior to nebulization, with a larger volume of an aqueous vehicles such as 1-5 mL of marketed sterile saline to form a two-phase liquid-liquid suspension, which can be administered effectively via a commercially available nebulizer. This invention requires the measurement of a small volume (0.05 to 0.5 mL) of the non-aqueous medicament concentrate from a multi-dose container that has all the problems previously addressed with multi-dose containers and dropper systems. Specifically these systems are inaccurate with respect to the amount of administered medicament and result in over or under dosage of the medicament.

In view of the disadvantages of the liquid solutions and suspensions and the costly process of the preparation of lyophilized product for oral inhalation, as well as the addition of soluble matrix ingredients that are unnecessary and unwanted, it becomes highly desirable to develop a dosage form to overcome the aforementioned disadvantages for liquid dosage forms and lyophilized products. The dosage form of the current invention is a dried solid or semi-solid uniform dispersion of medicament particles impregnated in or deposited on an inert supporting material. The dosage form does not contain or may contain a low level of preservative and is able to provide microbial, chemical, and physical stability for the medicament formulation. In addition, because the dosage form does not contain liquid and it is light in weight and volume and less costly to prepare.

### OBJECTS OF THE INVENTION

It is an object of the invention to provide a system for the delivery of a pharmaceutically active substance to the respiratory system through a liquid for oral-or nasal inhalation by a nebulizer.

It is a further object of the invention to provide a system for the delivery of a pharmaceutically active substance to the respiratory tract by a nebulizer where the pharmaceutically active substance is free of excipients that are not therapeutically necessary.

It is a further object of the invention to provide a system for the delivery of a pharmaceutically active substance to the respiratory tract by a nebulizer where the pharmaceutically active substance is administered to the patient in a repeatedly reliable, accurate dosage.

### SUMMARY OF THE INVENTION

The present invention provides compositions, method of preparation, and method of use for a pharmaceutical dosage unit comprising one or more therapeutically effective amounts of pharmaceutically active ingredients for oral or nasal inhalation. The pharmaceutically active ingredient is uniformly impregnated in or deposited on an inert supporting material in a dried solid or semi-solid state. The inert supporting material must maintain its integrity, when wet. The dosage form may be reconstituted with sterile water or sterile saline solution prior to administering to a patient via a nebulizer.

The present invention further provides a dosage form for oral or nasal inhalation of a pharmaceutically active ingredient that is impregnated in or deposited on an inert supporting material in a dried solid or semi-solid state. An inert supporting material is a material that does not contribute to the pharmaceutical activity of the dosage form.

The present invention further provides a pharmaceutical delivery system for nasal or oral inhalation for respiratory administration through nebulization.

In one aspect, the pharmaceutical active ingredients to be delivered using the dosage form of the present invention include any drugs that may be suitable for respiratory inhalation therapy to provide local or systemic drug delivery, such as, but not limited to, chemotherapy, treatment of pain, antibiotics, and those pharmaceutically active ingredients for treatment of respiratory disorder such as, but not limited to, adenosine triphosphate, acefylline, amlexanox, á-antitrypsin, n-acetylcysteine, albuterol sulfate, ambuphylline, ambroxol hydrochloride, amiloride, aminophylline, atropine sulfate, bambuterol, banifylline, beclomethasone dipropionate, bevonium methyl sulfate, bitolterol mesylate, bromhexiene hydrochloride, broxaterol, budesonide, carbuterol, choline theophyllinate, clenbuterol, cloprenaline, colistin, cromolyn sodium, dexamethasone sodium phosphate, dioxethedrine, doxapram, doxofylline, doxorubicin, dornase alpha, dyphylline, ephedrine, epozinol, etafedrine, etamiphylline, ethylnorepinephrine, etofylline, fenoterol, fenspiride, fentyl, formoterol fumarate, flunisolide, fluticasone propionate, flutropium bromide, formoterol tartrate, furosemide, genistein, gentamycin, glycopryrrolate, guaifenesin, heparin, hexoprenaline, hydrocortisone, iodionated glycerol, ibudilast, insulin, ipratropium bromide, isoetharine hydrochloride, isoetharine mesylate, isoproterenol hydrochlride, israpafant, ketotifen, levabuterol hydrochloride, levmetamfetamine, lidocaine, likura, lodoxaminde trometanol, mabuterol, magnesium sulfate, medibazine, metaproperenol sulfate, ipratropium bromidemethoxyphenamine, methylprednisolone, milrinone, mometasone furoate, montelukast, morphine, nedocromil sodium, neitilmicin, normal saline, oxatomide, orciprenaline sodium, oxitropium bromide, pentamidine, peptides, phenylbtyrate, pirbuterol hydrochloride, polymixin B, pranlukast, prednisone, procaterol hydrochloride, protokylol, proxyphylline, racemic epiephrine, ramatroban, recombinant human D-nase, reproterol hydrochloride, rimiterol hydrobromide, salmeterol xinaforte, seratrodast, sodium bicarbonate, sodium cromoglycate, soterenol, supatast tolsylate, terbutaline sulfate, theobromine, 1-theobromineacetic acid, theophylline, tiaramide, tiotropium bromide, pirbuterol acetate, levalbuterol hydrochloride, salmeterol xinafoate, flunisolide propionate, tobramycin, traxanox, tretoquinol, triamcinolone acetonide, tulobuterol hydrochloride, tranilast, uridine triphosphate, zafirlukast, zileutin, budesonide, beclomethasone dipropionate, fluticosone; antiinfective agents such as, but not limited to, tetracycline, penicillin, trimethoprim, ampicillin, amoxicillin, doxycycline, erythromycin, clarithromycin, azithromycin, dirithromycin, either alone or in combination with each other or beta-lactamase inhibitors such as clavulanate. Preferably the dosage of the antibiotics contained in the dosage form is no more than 50% of the usual effective dosage and more preferably is 10 to 30% of the usual effective dosage. Other medicaments to be delivered for systemic absorption include, but are not limited to, peptides and proteins such as insulin.

The present invention includes pharmaceutical compositions for the dosage form. In one feature, the dosage form contains a supporting material, which is an inert material used to impregnate or deposit the pharmaceutically active ingredient and formulation excipients, and has characteristics of:

a). does not interact with the pharmaceutically active ingredient and the formulation excipients;

b). is capable of adsorbing and retaining the medicament particles during the shelf life of product;

c). is able to release the pharmaceutically active ingredient substantially immediately after reconstituting with a sterile solution for inhalation;

d). does not disintegrated into pieces that might move together with the fine mists during nebulization, in other words maointains its integrity when wet and is neither water-soluble nor water-dispersible;

e). after reconstitution, does not substantially adsorb water in a way to reduce volume of solution available for nebulization; and

f). can be sterilized, if necessary.

The supporting material may be made from natural or synthetic polymers, woven or non-woven fabrics, paper, inorganic materials such as foil and the combination thereof, in a single or multi-layer laminations in a form of a sheet or strip or film or membrane or a cup, a well, or a sponge-like form. The polymer is selected from polyvinyl acetate, water-insoluble polyvinylalcohol, polyethylene oxide, polyethylene, ethylene-vinyl acetate copolymers, polypropylene, polybutylene, polyisobutylene, polystyrene, polyester, polyethylene terephthalate, nylon, polyacrylic, rayon, cellulose acetate, cellulose nitrate, polyethersulfone, polysulfone, polytetrafluoroethylene, polyvinylidene fluoride, glass micro fiber. The paper may be kraft paper or filter paper or paper made with cellulosic fiber selected from the group consisting of wood pulp fibers, cotton fibers, hemp fibers, jute fibers, and mixtures thereof. The paper may be silicone or wax-coated. The supporting material may be made from a single layer or may be a laminated or impregnated multi-layer of polymers and/or fabrics and/or paper and/or cotton and/or rayon and/or gauze and/or inorganic materials. The composition, size and thickness of the supporting material is determined such as the supporting material is able to adsorb or retain the formulation concentrate while the formulation concentrate is impregnated in and/or deposited on the supporting material and capable of holding the dried formulation which is impregnated in or deposited on the supporting material during the shelf life of the product.

In another embodiment of the composition of the dosage form, the dosage form may contain pharmaceutical excipients that can either improve the stability of the dosage form or provide comfort during administration. Such excipients include tonicity adjusting agents, pH adjusting or buffering agents, stabilization agents, anti-microbial preservatives, dispersing and wetting agents and pharmaceutical solvents.

Tonicity-adjusting agents are used to enhance the overall comfort to the patient upon administration of the reconstituted dosage form. It is preferred to adjust the osmolality of the reconstituted inhalation solution to about 275 to 305 (range 254 to 325) mOsm/Kg. Tonicity-adjusting agents for inhalation use are sodium chloride, dextrose, lactose, sodium phosphate, sorbitol, mannitol and sucrose or combination thereof at a concentration to generate an isotonic solution after the dosage form is reconstituted with 1 to 10 ml of sterile water for nebulization. The addition of sugars such as dextrose, lactose and sucrose adds stickiness and adherent characteristics to the formulation so that the dried-medicament and formulation excipients can be better retained on the supporting material after the formulation concentrate is dried. The tonicity-adjusting agent can also function as a particle partition agent to reduce particle size of the pharmaceutically active ingredient after the formulation is impregnated in or deposited on the supporting material and to assist in dissolution or dispersion of pharmaceutically active ingredient particles upon reconstitution with the pharmaceutical solvent. Alternately, the dosage form may be formulated without the addition of a major tonicity-adjusting agent. The desired tonicity of the dosage form is achieved by reconstituting with a sterile isotonic saline solution.

Next pH adjusting or buffering agents are used to adjust or maintain the pH of pharmaceutical dosage form to a desired range for the following reasons: (1) To provide an environment for a better product stability that pharmaceutical active ingredient may express a better chemical stability within certain pH range, (2) to provide better comfort for the patient at administration. Extreme pH may create irritation and/or discomfort to the site of administration, and (3) to provide a pH range for better anti-microbial preservative activity. Some preservatives such as benzoic acid and sorbic acid require a lower pH for a better anti-microbial activity. Dosage forms of the present invention may be formulated with one or more pharmaceutically acceptable pH adjusting or buffering agents so that, after reconstitution, the desired pH is between about 3 to about 8. Pharmaceutically acceptable pH-adjusting and buffering agents are selected from, but not limited to, hydrochloric acid, sulfuric acid, nitric acid, acetic acid, phosphoric acid, fumaric acid, citric acid, tartaric acid, maleic acid, succinic acid, ammonia solution, ammonium carbonate, sodium borate, sodium carbonate, triethanolamine, trolamine and sodium hydroxide.

Stabilizing agents are antioxidant and chelating agents that are capable of inhibiting oxidation reaction and chelating metals, respectively, to improve stability of pharmaceutically active ingredient and excipients. Dosage forms of the present invention may be formulated with one or more pharmaceutically acceptable stabilization agents at a concentration suitable for the intended pharmaceutical applications, and may be selected from, but not limited to, chelating agents such as EDTA and its sodium salt, citric acid and sodium citrate, anti-oxidation agents such as Vitamin E, ascorbic acid, ascorbyl palmitate, butylated hydroxyanisole, butylated hydroxytoluene, hypophosphorous acid, monothioglycerol, propyl gallate, sodium bisulfite, sodium metabisulfite, sodium formaldehyde sulfoxylate, and thiourea.

The addition of a stabilizing agent to a dosage form of the present invention can improve stability of the pharmaceutical active substance and prolong the shelf life.

Although a stabilizing agent may be added to improve stability of the dosage form, it is clear that the dosage form of the present invention in which the pharmaceutically active ingredient is dispersed in a dried form and therefore, may need no stabilizing agents or only require a lower level of stabilizing agent in comparison to that required for a liquid dosage form which, in general, requires a higher level of stabilizing agent to protect an unstable pharmaceutically active ingredient or excipients from being oxidized.

Anti-microbial preservative agents are used in pharmaceutical preparations to inhibit the growth of microorganisms. Dosage forms of the present invention may be formulated with one or more pharmaceutically acceptable anti-microbial preservatives at suitable concentrations to prevent microbial growth. Examples of pharmaceutically acceptable preservatives suitable for oral or nasal inhalation include, but are not limited to, parabens, benzalkonium chloride, benzethonium chloride, benzoic acid, sorbic acid or potassium sorbate, benzyl alcohol, cetylpyridinium chloride, chlorobutanol, phenol, phenylethyl alcohol, phenylmercuric nitrate, and thimerosal.

Although an anti-microbial preservative may be added for better stability of the dosage form, preservative(s) often are toxic and/or cause hypersensitivity reactions in patients. It is clear that the dosage form of the present invention in which the pharmaceutically active ingredient is dispersed in a dried form, may need no anti-microbial preservative or only require a lower level of preservative in comparison to what is required for a liquid dosage in the prevention of microbial growth. The elimination of an anti-microbial preservative from a pharmaceutical formulation reduces the potential risk of hypersensitivity reaction.

Wetting or dispensing agents are used to increase wettability and assist in dispersing of water insoluble or poorly water soluble particles. For water insoluble and poorly water soluble medicaments, the addition of one or more wetting or dispersing agents to the dosage formulation of the present invention can help the release of the impregnated pharmaceutical active ingredient particles from the supporting material into the reconstituted solution and can help the dispersion of the particles to form a fine suspension. Examples of pharmaceutically acceptable wetting and dispersing suitable for oral or nasal inhalation agents are, poloxamers, oleic acid and its salts, lecithin and hydrogenated lecithin, sorbitan fatty acid esters oleyl alcohol, phospholipids including but not limited to phosphatidylglycerol, phosphatidylcholine and others, polyoxyethylene fatty alcohol ethers, polyoxypropylene fatty alcohol ether, polyoxyethylene fatty acid ester, glycerol fatty acid esters, glycolipid such as sphingolipid and sphingomyelin, polyoxyethylene glycol fatty acid ester, polyol fatty acid esters, polyethylene glycol glycerol fatty acid esters, polypropylene glycol fatty acid esters, ethoxylated lanolin derivatives, polyoxyethylene fatty alcohol, polyoxyethylene sorbitan fatty acid esters, polyoxyethylene stearate, propylene glycol alginate, dilauryldimethylammonium chloride, D-α-tocopheryl-PEG 1000 succinate, Polyoxy 40 stearate, polyoxyethylene-polyoxypropylene block copolymers, polyoxyethylene vegetable oils, fatty acid derivatives of amino acids, glyceride derivatives of amino acids, benzalkonium chloride, bile acids.

Pharmaceutical solvents are used to dissolve or disperse pharmaceutically active medicaments and excipients. The solvent may be aqueous or non-aqueous. A dosage form of the present invention may be formulated with one or a mixture of more than one pharmaceutically acceptable solvent and is selected from, but not limited to, glycerol, propylene glycol, polyethylene glycol, polypropylene glycol, ethyl alcohol, isopropyl alcohol, water, mineral oil, peanut oil, and corn oil. The pharmaceutical solvents may be used to prepare the formulation concentrate as well as used for reconstitution of the dosage form of the present invention. Pharmaceutically acceptable solvents such as water, ethyl alcohol, isopropyl alcohol are evaporable and are usually used to dissolve or disperse the medicament and excipients in the formulation concentrate. Glycerol, propylene glycol and polyethylene glycol are co-solvents and are used to assist in solubilization of water insoluble or poorly water soluble medicaments in the formulation concentrate. Pharmaceutically acceptable reconstituting solvents such as sterile water for injection, sterile normal saline solution, sterile phosphate buffer solution and sterile 5% dextrose solution are used for reconstitution of the dosage form of the present invention to form a solution or a fine particle suspension of pharmaceutically active ingredient prior to oral or nasal inhalation via a nebulizer. The reconstituting solvents may be packaged in individual ampoules or unit dose plastic containers for unit of use or may be packaged in large volume sterile container from which a specific volume of the reconstituting solvent can be withdrawn without contaminating the solvent.

Another aspect of the present invention is a method of preparation of the dosage form. The pharmaceutically active ingredients may be dissolved or dispersed as fine particles in water or other pharmaceutical solvents or a mixture thereof with other excipients to form the formulation concentrate.

In one embodiment for the preparation of formulation concentrate, it is preferred that the water soluble pharmaceutically active ingredients be dissolved in water together with a suitable amount of pH-adjusting agents to the desired pH (3 to 8). A excipient such as dextrose, lactose, mannitol, or sorbitol may be added to help adherence of drug to the supporting material after drying. A stability agent and/or a preservative at a low level may be added to the formulation concentrate, if needed, for a better chemical and microbial stability of the dosage form. A portion of water in the formulation concentrate may be replaced with ethyl alcohol for a faster solvent evaporation.

In another embodiment for the preparation of formulation concentrate for water insoluble or poorly water-soluble pharmaceutically active ingredients, the formulation concentrate may be prepared by dissolving the pharmaceutically active ingredients in a pharmaceutical solvent mixture containing water and an organic solvent such as ethyl alcohol, isopropyl alcohol, propylene glycol and/or polyethylene glycol or a combination thereof together with other excipients such as dextrose, lactose, and sucrose. A stabilization agent or preservative may be added at a low level, if needed, for a better chemical and microbial stability of the product. A pH-adjusting agent may be added to adjust pH to desired range. A wetting and dispersing agent may be added to the formulation concentrate for the reason that it increases the wettability of the poor water soluble particles and helps in releasing the drug particles from the supporting material at constitution. The wetting or dispersing agent also helps in forming a better dispersion of drug particles in the reconstituted solution.

In another embodiment including an alternative procedure for the preparation of formulation concentrate for water insoluble or poorly water-soluble pharmaceutically active ingredients, the water insoluble pharmaceutically active ingredient particles in micronized particle size of smaller than 10 microns, preferably smaller than 5 micron, may be dispersed in an aqueous solution. A excipient such as dextrose, lactose, or sucrose may be added to adhere the medicament to the supporting material after the dosage form is dried. Like the formulation described above, a stabilization agent or preservative may be added at a low level, if needed, for better chemical and microbial stability of the product. A pH-adjusting agent may be added to adjust pH to a desired range. A wetting and dispersing agent may be added to the formulation concentrate for the reason that it increases the wettability of the poor water soluble particles and helps in releasing the drug particles from the supporting material at constitution. The wetting or dispersing agent also helps in forming a better dispersion of drug particles in the reconstituted solution.

The formulation concentrate may or may not contain a tonicity-adjusting agent. A tonicity-adjusting agent may be added in an amount, when the dosage form is reconstituted with water, to generate a solution of tonicity in the range of (254 to 325 mOsmol/Kg). In case that a major tonicity-adjusting agent is not included in the formulation concentrate, the adjustment of the tonicity of the reconstituted solution is achieved by reconstituting the dosage form with a normal saline solution or a 5% Dextrose solution.

The formulation concentrate is then transferred onto supporting material by means of spraying or pipeting or a liquid dispensing device a specific volume of the formulation concentrate onto supporting material such as a piece of filter paper or into a well or cup of a pre-determined size.

The supporting material on which the formulation concentrate is deposited is then dried with or without a raised temperature and/or with or without a vacuum force and/or with or without a nitrogen gas flow to remove the evaporable solvent. After most of the evaporable solvent is removed, the medicament formulation forms a dry powder impregnation in or deposition on the supporting material. The supporting material containing the dried medicament is then cut or perforated into a size containing a therapeutically effective amount of the pharmaceutical active ingredient for unit dose uses. The dosage form is ready for further package.

Alternatively, a volume of formulation concentrate solution equivalent to a therapeutically effective amount of one unit dose may be transferred to a predetermined size of supporting material for unit dose use. The supporting material containing the medicament is then dried to obtain the final dosage form without further downsizing. Such a predetermined size of supporting material may be a piece of filter paper, film, a cup or well.

The dosage form may be sterilized using sterilization procedure such as radiation known in the prior art. The dried supporting material-medicament may be wrapped and sealed with foil as a card containing individual unit doses or as individual dosage units. Multi-unit doses may be packaged in a container or a dispensing device from which a single unit dose may be dispensed. An alternate method of transferring the formulation concentrate to the supporting material is to dip the supporting material in the formulation concentrate until the supporting material is saturated with the formulation concentrate and is then dried.

Another aspect of the present invention is method of use and administration of the dosage form. Prior to administration, the dose unit is reconstituted with a specific volume of, either sterile water or sterile normal saline solution or sterile dextrose solution depending on the amount of tonicity-adjusting agent added in the formulation concentrate. The solution is then shaken or agitated to release pharmaceutically active ingredient particles from the supporting material. The agitation may be done manually or with the aid of a mechanical mean such as a sonication force or an atomized air. A solution or a suspension containing finely dispersed medicament particles is formed. The resulting solution or suspension is then administered to a patient using a nebulizer for nasal or oral inhalation. The reconstitution of solution may take place in the drug reservoir of a nebulizer or may be prepared in a separate container and then transferred to the drug reservoir of a nebulizer prior to oral inhalation. The inert supporting material, which may be a piece of filter paper or film or strip or sponge-like or a small plastic well and is not dissolved nor disintegrated in the reconstituted solution, may remain in the drug reservoir during nebulization or may be removed from the reconstituted solution or suspension after the drug is dissolved or released from the supporting material and prior to administration.

The present invention further provides a pharmaceutical delivery system for nasal or oral inhalation for respiratory administration through nebulization, which comprises:

(a) a water-tight container having an inlet for receiving a jet of compressed air or containing a plate capable of vibrating at ultrasonic frequency, and an opening through which a nebulizing mist exits the container;

(b) a pharmaceutical unit dosage form comprising an inert supporting material, which when wet maintains its integrity, on which is deposited or in which is impregnated a therapeutically effective amount of at least one pharmaceutically active ingredient capable of oral inhalation wherein the inert supporting material is capable of absorbing or retaining the at least one pharmaceutically active ingredient and of releasing the at least one pharmaceutically active ingredient substantially immediately after being reconstituted with a reconstituting solvent;

(c) a reconstituting solution comprising the reconstituting solvent in contact with the inert supporting material impregnated with or deposited on the at least one pharmaceutically active ingredient to form a solution or suspension of the pharmaceutically active ingredient in the reconstituting solvent; and

(d) means for introducing compressed air or ultrasonically vibrated air through the inlet into the water-tight reservoir to nebulize the solution or suspension of the at least one pharmaceutically active ingredient capable of oral inhalation in the reconstituting solvent to form a nebulizing mist which exits the water-tight container into the nose or mouth of a patient.

The pharmaceutical delivery system may further comprise:

(e) means located within the water-tight container above the ultrasonic vibrating plate or above the compressed air inlet to distribute the vibrated frequency air or compressed air introduced throughout the reconstituted solution or suspension of the at least one pharmaceutically active ingredient in the reconstituting solvent to form the nebulizing mist.

The reconstituting solvent that forms a solution or a suspension with the at least one pharmaceutically active ingredient preferably is selected from the group consisting of water, aqueous saline solution, aqueous dextrose solution, or an aqueous buffer solution preferably buffered at a pH of 3 to 8.

### BRIEF DESCRIPTION OF THE DRAWINGS

FIG. 1 is a perspective drawing of a preferred version of the pharmaceutical delivery system for providing a mist containing a pharmaceutically active ingredient for nasal or oral inhalation.

FIG. 2 is a drawing of a pharmaceutical unit dosage form containing an inert supporting material, which when wet maintains its integrity, on which is deposited or in which is impregnated a pharmaceutically active ingredient, said pharmaceutical unit dosage form providing the pharmaceutically active ingredient in the pharmaceutical delivery system.

FIG. 3 is a drawing of ampoules containing a reconstituting solvent which when released from the ampoules and contacted with the pharmaceutically active ingredient in the pharmaceutical dosage form produces a reconstituted solution or suspension of the pharmaceutically active ingredient.

### DETAILED DESCRIPTION OF THE DRAWINGS

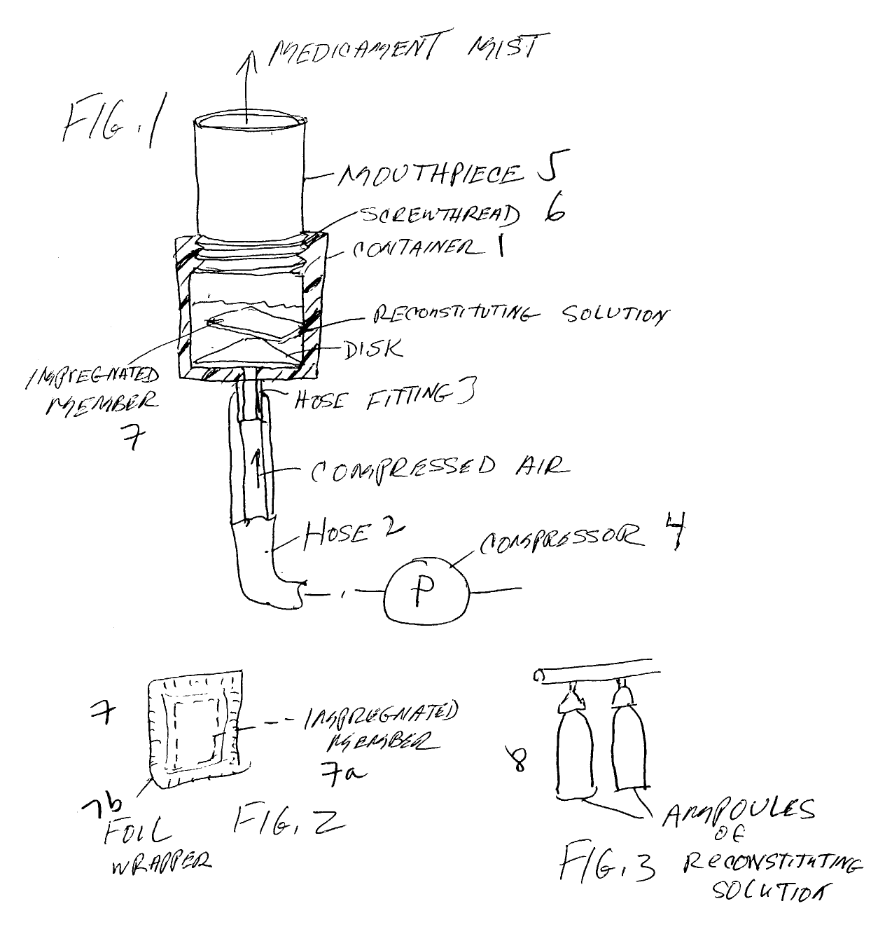

The pharmaceutical delivery system in FIG. 1 includes a water-tight container or drug reservoir 1 capable of holding a reconstituting solvent or a reconstituted solution or suspension of the pharmaceutically active ingredient. Attached to one face of the water-tight container is a hose 2 for providing compressed air to the water-tight container through hose fitting 3 which is an inlet into the container. The compressed air is provided by compressor 4 to the inlet. Attached to another face of the water-tight container is a mouthpiece 5 which may be secured to an opening in the container by a screw thread 6. Inside the container the pharmaceutical unit dosage form 7 may be placed. The pharmaceutical dosage form comprises an inert supporting material, which when wet maintains its integrity, on which is deposited or in which is impregnated, a therapeutically effective amount of at least one pharmaceutically active ingredient capable of administration by oral or nasal inhalation. The pharmaceutically active ingredient is released from the inert supporting material when contacted with a reconstituting solvent. The reconstituting solvent must be capable of forming a solution or a suspension of the pharmaceutically active ingredient.

The reconstituting solvent is added to the water-tight container holding the pharmaceutical unit dosage form and compressed air is generated in the compressor 4, passed through hose 2 and hose fitting 3 into the water-tight container to provide a reconstituted solution comprising a pharmaceutically active ingredient suitable for oral or nasal inhalation in the form of a medicament mist. The medicament mist exits the water-tight container through the opening communicating with the mouthpiece and then through the mouthpiece itself to reach the nose or mouth of a patient so as to administer to the patient the pharmaceutically active ingredient by oral or nasal inhalation.

Alternatively in another feature of the present invention, in the pharmaceutical delivery system the pharmaceutical unit dosage 7 may be placed and the reconstituting solvent added to a separate vessel to form the reconstituted solution comprising a pharmaceutically active ingredient suitable for oral or nasal inhalation. The reconstituted solution comprising the pharmaceutically acceptable active ingredient dissolved or dispersed therein, may then be directly added to the water-tight chamber to form the medicament mist.

The pharmaceutical unit dosage form that is FIG. 2 includes an inert support 7a which when wet maintains its integrity on which is deposited or in which is impregnated the pharmaceutically active ingredient. The inert support may further comprise a foil wrapping 7b which surrounds the inert support 7a with an air-tight and water-tight envelope to enable stable storage of the pharmaceutically active ingredient. When ready for use the foil wrapping may be removed from the pharmaceutical unit dosage form and the inert support 7a is then contacted with a reconstituting solvent to form a reconstituted solution comprising a pharmaceutically active ingredient suitable for oral or nasal inhalation. The inert supporting material must be capable of absorbing or retaining the pharmaceutically active ingredient and of releasing the pharmaceutically active ingredient substantially immediately after being reconstituted with a reconstituting solvent.

The ampoules 8 that are shown in FIG. 3 are conventional ampoules well known in the art. The ampoules have an air-tight and water-tight seal and contain a sterile reconstituting solvent such as water or saline solution. When the seal is broken the sterile reconstituting solution is released from the ampoules and directly contacted with the inert supporting material in which the pharmaceutically active ingredient is absorbed or at least retained in the pharmaceutical unit dosage form described in FIG. 2 to form a reconstituted solution of the pharmaceutically active ingredient.

### EXAMPLES

The present invention can be demonstrated more specifically with reference to the following examples, that are given for illustration of the present invention and are not intended to be limiting thereof.

### Example 1

### Albuterol Sulfate for Oral Inhalation

Albuterol is a relatively selective beta 2-adrenergic bronchodilator. The pharmacologic effects of beta-adrenergic agonist drugs are at least in part attributable to stimulation through beta-adrenergic receptors of intracellular adenyl cyclase, the enzyme that catalyzes the conversion of adenosine triphosphate (ATP) to cyclic-3′,5′-adenosine monophosphate (cyclic AMP). Increased cyclic AMP levels are associated with relaxation of bronchial smooth muscle and inhibition of release of mediators of immediate hypersensitivity from cells, especially from mast cells. Albuterol has been used for the relief of bronchospasm in patients with reversible obstructive airway disease and acute attacks of bronchospasm. Albuterol is available in dosage forms such as metered dose pressurized inhaler, dry powder inhaler and solutions for inhalation. The marketed solutions for inhalation include albuterol sulfate 0.5% and 0.083%. The 0.5% solution is required to be diluted with sterile normal saline solution and is formulated with a antimicrobial agent. It is packaged in a container for multi-dose uses. The 0.083% solution requires no dilution prior to administration. It has been formulated with an antimicrobial preservative agent in a container for multi-dose uses. Recently, due to the potential microbial growth problems with the 0.083% solution, a preservative-free, sterile unit dose packaged in low density polyethylene containers has become available.

This example provides a formulation of albuterol sulfate using the delivery system of the present invention. The formulation contains no preservative and requires no sterilization nor requires preparation under aseptic conditions.

Typical Formulation and Procedure:

Albuterol Sulfate Formulation Concentrate to be Applied onto a Supporting Material

| | % RANGE | TYPICAL FORMULATION |
|---|---|---|
| INGREDIENT | w/v | mg/100 mL |
| Albuterol Sulfate | 1.0-5.0 | 3000 |
| Dextrose | 3.0-15.0 | 5000 |
| Diluted. Sulfuric | For pH adjust to 3-5, | qs |
| acid | if needed | |
| Purified Water | Qs to 100 | Qs to 100.0 ml |

Procedure for Preparation of Dosage Unit:

a. Preparation of formulation concentrate: Albuterol sulfate and dextrose are dissolved in purified water. Adjust the pH of the solution to 3 to 5, if needed, and bring to desired volume with purified water.

b. Filter the formulation concentrate though a 0.45 micron or smaller sterile filter.

c. Spray the formulation concentrate solution evenly onto a supporting material (cellulose filter paper) of approximately 1000 cm². Dry the supporting material at approximately 25 to 50° C. until the material is practically dried. Apply air flow, or nitrogen gas flow, if needed, to facilitate the drying process.

d. After the dosage form is dried, it may be perforated or downsized to a size of approximately 1 cm².

e. Each dosage unit so obtained contains 3 mg of albuterol sulfate.

f. The dosage units may be further wrapped individually with aluminum foil. The perforated dosage units may be packaged in a dispensing device which is able to dispense one dosage unit.

Method of Use and Administration:

Prior to oral inhalation, one unit dose is placed in the drug reservoir of a nebulizer. Approximately 3-5 ml of sterile saline solution is added to the drug reservoir. Rotate the device horizontally in a way so the solution in the drug reservoir is swirled and the dosage unit is emerged in the solution. Continue rotating the device for about 1 minute. Turn on the atomized air or ultrasonic force to generate the mist for oral inhalation. Continue generating the mist until the desired amount of medicament is inhaled by the patient.

### Example 2

### Budesonide for Oral Inhalation

Budesonide is a an anti-inflammatory corticosteroid that exhibits potent glucocorticoid activity and weak mineralocorticoid activity. The precise mechanism of corticosteroid actions on inflammation in asthma is not known. Corticosteroids have been shown to have a wide range of inhibitory activities against multiple cell types (e.g., mast cells, eosinophils, neutrophils, macrophages, and lymphocytes) and mediators (e.g., histamine, eicosanoids, leukotrienes, and cytokines) involved in allergic and non-allergic-mediated inflammation. These anti-inflammatory actions of corticosteroids may contribute to their efficacy in asthma. Budesonide is commercially available as a dry powder inhalation (Pulmicort Turbuhaler) and a suspension for oral inhalation (Pulmicort Respulse 0.25 mg and 0.5 mg) via nebulization. The Respulse is available in sterile unit dose packaged in low density polyethylene container.

This example provides a formulation of albuterol sulfate using the delivery system of the present invention. The formulation contains no preservative and requires no sterilization nor requires preparation under aseptic conditions.

Typical Formulation and Procedure:

Budesonide Formulation Concentrate to be Applied onto a Supporting Material

| | % RANGE | TYPICAL FORMULATION |
|---|---|---|
| INGREDIENT | w/v | mg/mL |
| Budesonide, | 0.5-1.5 | 8.3 |
| micronized (<5 | | |
| micron, preferably <3 | | |
| microns) | | |
| Dextrose | 5-20 | 100 |
| Tween 80 | 0.1-1.0 | 5.0 |
| (polysorbate 80) | | |
| Purified Water | Qs to 100% | Qs to 1.0 ml |

* Budesonide may be micronized in powder state or be micronized in liquid state using an atomized air technology or other means.

Procedure for Preparation of Dosage Unit:

a. Preparation of formulation concentrate:

Tween 80 and dextrose are dissolved in purified water. Filter the solution through a 0.45 or smaller filter. Disperse micronized budesonide in the solution and stir to form uniform dispersion.

b. Transfer 30 μL of the formulation concentrate into a small polystyrene well of approximately 1 cm in diameter and approximately 1 mm in dept. Let the solution dry in the air or with the aid of air or nitrogen gas flow or with a vacuum.

c. Each well contains about 0.25 mg of budesonide. The well may be wrapped individually in foil to protect the formulation.

A polystyrene card molded with multiple small wells may be used for preparation instead of individual wells.

Method of Use and Administration:

Prior to oral inhalation, one unit dose is removed from the package and placed in the drug reservoir of a nebulizer. Approximately 2-4 ml of sterile saline solution is added to the drug reservoir. Rotate the device horizontally in a way so the solution in the drug reservoir is swirled and the dosage unit is emerged in the solution. Continue swirling the device for about 1 minute. Turn on the atomized air or ultrasonic force to generate the mist for oral inhalation. Continue generating the mist until the desired amount of medicament is inhaled by the patient.

### REFERENCES

| | | |
|---|---|---|
| WO99/44594 | March 1999 | Sonoke, S. et al |
| US5192528 | March 1993 | Radhakrishman, R. et al |
| US6241969 | June 2001 | Saidi, Z. et al |
| US6161536 | December 2000 | Redmon, M. A., et al |
| WO01/47491 | December 1999 | Coifman, R. E., et al |
| US6187765 | February 2001 | Harris, et al |
| US5993781 | November 1999 | Snell, et al |

### OTHER REFERENCES

Dipiro, J. T. “A Pathophysiologic Approach, Fourth Edition, Appleton and Lange, Stamford Conn. 1999

Editors: Kelly, H. W. and Kamada, A. K., Chapter 24 Asthma pages 430-460

Editors: Konzem, S. L., Stratton, M. A., Chapter 25, Chronic Obstructive Lung Disease

Editors: Gal. P., Watling, S. M., Shaffer, C. L., Chapter 26, Respiratory Distress Syndromes, pages 478-493

Remington: The Science and Practice of Pharmacy, 20^{th} Edition, Lippincott, Williams & Wilkins, Baltimore, Md.

Editors: Wai-Yip Lee, T., Tobinson, J. R., Chapter 47 Controlled-Release Drug Delivery Systems, page 919 (liposomes)

Editors: Sciarra, J. J., Sciarra, C. J., Chapter 50 Aerosols, pages 963-964, 974-979

Editors: Vadas, E. R., Chapter 52 Stability of Pharmaceutical products pages 993-994

Editors: Rabinow, B. E., Roseman, T. J., Chapter 54 Plastic Packaging Material pages 1009-1010 (thermoplastics), 1012-1013 (sterilization)

Approved Drug Products 21^{st} Edition, U.S. Department of Health and Human Service

Parfitt, K., Editor “Marindale=The complete Drug Reference” thirty-second edition, Pharmaceutical Press, London, UK 1999

Physicians' Desk Reference 52 Edition, Medica; Economics, Montvalle, N.J. (Which page or what product in the PDR?)

What is claimed is:

1. A pharmaceutical delivery system for nasal or oral inhalation for respiratory administration through nebulization, which comprises:
(a) a water-tight container having an inlet for receiving a jet of compressed air or containing a plate capable of vibrating at ultrasonic frequency, and an opening through which a nebulizing mist exits the container;
(b) a pharmaceutical unit dosage form comprising an inert supporting material, which when wet maintains its integrity, on which is deposited or in which is impregnated a therapeutically effective amount of at least one pharmaceutically active ingredient capable of oral inhalation wherein the inert supporting material is capable of absorbing or retaining the at least one pharmaceutically active ingredient and of releasing the at least one pharmaceutically active ingredient substantially immediately after being reconstituted with a reconstituting solvent;
(c) a reconstituting solution comprising the reconstituting solvent in contact with the inert supporting material impregnated with or deposited on the at least one pharmaceutically active ingredient to form a solution or suspension of the pharmaceutically active ingredient in the reconstituting solvent; and
(d) means for introducing compressed air or ultrasonically vibrated air through the inlet into the water-tight reservoir to nebulize the solution or suspension of the at least one pharmaceutically active ingredient capable of oral inhalation in the reconstituting solvent to form a nebulizing mist which exits the water-tight container into the nose or mouth of a patient.

2. The pharmaceutical delivery system defined in claim 1 which further comprises:
(e) means located within the water-tight container above the ultrasonic vibrating plate or above the compressed air inlet to distribute the vibrated frequency air or compressed air introduced throughout the reconstituted solution or suspension of the at least one pharmaceutically active ingredient in the reconstituting solvent to form the nebulizing mist.

3. The pharmaceutical delivery system defined in claim 1 wherein the reconstituting solvent forming a solution or a suspension with the at least one pharmaceutically active ingredient is selected from the group consisting of water, aqueous saline solution, aqueous dextrose solution, or an aqueous buffer solution buffered at a pH of 3 to 8.

4. A pharmaceutical composition in unit dosage form which comprises an inert supporting material on which is deposited or in which is impregnated a therapeutically effective amount of at least one pharmaceutically active ingredient capable of oral inhalation wherein the inert supporting material is capable of absorbing or retaining the at least one pharmaceutically active ingredient and of releasing the at least one pharmaceutically active ingredient substantially immediately after being reconstituted with a reconstituting solvent.

5. The pharmaceutical composition in unit dosage form defined in claim 4 wherein the at least one pharmaceutically active ingredient capable of oral inhalation is impregnated in or deposited on the inert supporting material in a dried solid or semisolid state.

6. The pharmaceutical composition in unit dosage form defined in claim 4 wherein the inert supporting material comprises a natural or synthetic polymer, woven or non-woven fabric, paper, cotton, gauze, or a foil, or combinations thereof as a single or multi-layer lamination in a sheet, strip, film, membrane, cup, or well or as a sponge-like lamination.

7. The pharmaceutical composition in unit dosage form defined in claim 6 wherein the natural or synthetic polymer is selected from the group consisting of polyvinylacetate, water-insoluble polyvinylalcohol, polyethylene oxide, polyethylene, ethylene-vinyl acetate copolymer, polypropylene, polybutylene, polyisobutylene, polystyrene, polyester, polyethylene terephthalate, nylon, PVC, rayon, polyether sulfone, polysulfone, polytetrafluoroethylene, polyvinylidene fluoride, and glass microfiber, or a combination of more than one of said natural or synthetic polymer.

8. The pharmaceutical composition in unit dosage form defined in claim 6 wherein the paper is kraft paper coated with a silicone or a wax, filter paper, or, a paper made with cellulosic fiber.

9. The pharmaceutical composition in unit dosage form defined in claim 4 wherein the at least one pharmaceutically active ingredient capable of oral inhalation is formulated with at least one pharmaceutically acceptable excipient selected from the group consisting of a tonicity adjusting agent, a pH adjusting or buffering agent, a wetting and dispersing agent, a stabilization agent, and an antimicrobial agent and preservative, and a pharmaceutical solvent.

10. The pharmaceutical composition in unit dosage form defined in claim 9 wherein the tonicity adjusting agent is selected from the group consisting of sodium chloride, dextrose, Lactose, sucrose, mannitol, sorbitol, sodium phosphate, sodium bicarbonate, and an amino acid, or combinations thereof.

11. The pharmaceutical composition in unit dosage form defined in claim 9 wherein the pH adjusting or buffering agent is selected from the group consisting of hydrochloric acid, nitric acid, sulfuric acid, acetic acid, phosphoric acid, fumaric acid, citric acid, tartaric acid, succinic acid, aqueous ammonia solution, ammonium carbonate, sodium borate, sodium carbonate, and sodium hydroxide, or combinations thereof.

12. The pharmaceutical composition in unit dosage form defined in claim 9 wherein the wetting and dispersing agent is selected from the group consisting of a polysorbate, oleic acid, lecithin, sodium trioleate, tocopheryl polyethylene glycol 1000 succinate, a poloxamer, a phospholipid, a polyoxyethylene fatty alcohol ether, a polyoxypropylene fatty alcohol ether, polyoxyethylene fatty acid ester, a glycerol fatty acid ester, a glycolipid, polyoxyethylene glycol fatty acid ester, polyol fatty acid ester, polyethylene glycol glycerol fatty acid ester, polyol fatty acid ester, a polypropylene glycol fatty acid ester, an ethoxylated lanolin, a polyoxyethylene fatty alcohol, a polyoxyethylene sorbitan fatty acid ester, a polyoxyethylene stearate, propylene glycol alginate, a diaryldimethyl ammonium chloride, polyoxy 40 stearate, a polyoxyethylene polyoxypropylene block copolymer, a polyoxyethylene vegetable oil, a fatty acid derivative of an amino acid, a glycerol derivative of an acid, benzalkonium chloride and a bile acid or combinations thereof.

13. The pharmaceutical composition in unit dosage form defined in claim 9 wherein the stabilization agent is at least one chelating agent or antioxidant or combinations thereof.

14. The pharmaceutical composition in unit dosage form defined in claim 9 wherein the antimicrobial agent and preservative is selected from the group consisting of benzalkonium chloride, benzethonium chloride, benzyl alcohol, butyl paraben, cetyl pyridinium chloride, chlorobutanol, methylparaben, phenol, phenylethyl alcohol, phenylmercuric nitrate, and propylparaben or combinations thereof.

15. The pharmaceutical composition in unit dosage form defined in claim 9 wherein the pharmaceutical solvent is selected from the group consisting of glycerine, propylene glycol, polyethylene glycol, polypropylene glycol, ethyl alcohol, peanut oil, corn oil and water.

16. The pharmaceutical composition in unit dosage form defined in claim 4 packaged in an individual unit dose pouch or in an individual sealed cup or in a multi-dose pharmaceutically acceptable closed container, sealed pouch, or a dispensing device.

17. The pharmaceutical composition in unit dosage form defined in claim 4 which is sterilized or prepared under an aseptic condition.

18. A method for nasal or oral respiratory route administration of a pharmaceutically active ingredient capable of nasal or oral inhalation as a nebulizing mist to a patient which comprises the steps of:
(1) providing a pharmaceutical delivery system for oral inhalation through nebulization which comprises:
(a) a water-tight container in which mist is generated by a small plate vibrating at ultrasonic frequency, or by a jet of compressed air which is delivered to the container through an inlet, and an opening through which a nebulizing mist exits the container;
(b) a pharmaceutical unit dosage form comprising an inert supporting material, which when wet maintains its integrity, on which is deposited or in which is impregnated a therapeutically effective amount of at least one pharmaceutically active ingredient capable of oral inhalation wherein the inert supporting material is capable of absorbing or retaining the at least one pharmaceutically active ingredient and of releasing the at least one pharmaceutically active ingredient substantially immediately after being reconstituted with a reconstituting solvent;
(c) a reconstituting solution comprising the reconstituting solvent in the water-tight container in contact with the inert supporting material impregnated with the at least one pharmaceutically active ingredient to form a solution or suspension of the pharmaceutically active ingredient in the reconstituting solvent; and
(d) means for introducing ultrasonic frequency vibration or compressed air through the inlet into the water-tight container to nebulize the solution or suspension of the at least one pharmaceutically active ingredient capable of oral inhalation in the reconstituting solvent to form a nebulizing mist which exits the water-tight container through the opening into the nose or mouth of a patient;
(2) treating the inert supporting material on which is deposited or in which is impregnated with a therapeutically effective amount of at least one pharmaceutically active ingredient capable of oral inhalation with the reconstituting solvent to form a mixture, agitating or sonicating the mixture to form a solution or suspension of the pharmaceutically active ingredient in the reconstituting solvent thereby releasing the pharmaceutically active ingredient from the inert supporting material;
(3) introducing ultrasonic frequency vibration or compressed air through the inlet into the water-tight container to nebulize the solution or suspension of the pharmaceutically active ingredient in the reconstituting pharmaceutical solvent to form a nebulizing mist containing the pharmaceutically active ingredient; and
(4) administering the nebulizing mist containing the pharmaceutically active ingredient to the nose or mouth of the patient.

19. The method for nasal or oral respiratory route administration defined in claim 18 wherein according to step (2) the inert supporting material on which is deposited or in which is impregnated a therapeutically effective amount of at least one pharmaceutically active ingredient capable of oral inhalation is treated with the reconstituting solvent in the water-tight container to form a mixture.

20. The method for nasal or oral respiratory route administration defined in claim 18 wherein according to step (2) the inert supporting material on which is deposited or in which is impregnated a therapeutically effective amount of at least one pharmaceutically active ingredient capable of oral inhalation is treated with the reconstituting solvent in a container separate from the water-tight container to form a reconstituted solution or suspension which is then transferred to the water-tight container.

21. The method for nasal or oral respiratory route administration defined in claim 18 wherein following the release of the pharmaceutically active ingredient from the inert supporting material, the inert supporting material may be removed from the pharmaceutical delivery system through the opening.

22. A method of preparing a pharmaceutical composition in unit dosage form which comprises an inert supporting material, which when wet maintains its integrity, on which is deposited or in which is impregnated a therapeutically effective amount of at least one pharmaceutically active ingredient capable of oral inhalation wherein the inert supporting material is capable of absorbing or retaining the at least one pharmaceutically active ingredient and of releasing the at least one pharmaceutically active ingredient substantially immediately after being reconstituted with a reconstituting solvent, which comprises the steps of:
(a) dissolving or uniformly suspending a therapeutically effective amount of the at least one pharmaceutically active ingredient capable of oral inhalation in at least one pharmaceutical excipient and solvent to form a formulation concentrate;
(b) applying the formulation concentrate to the inert supporting material to deposit on or to impregnate in said inert supporting material the pharmaceutically active ingredient capable of oral inhalation;
(c) drying the inert supporting material on which is deposited or in which is impregnated the pharmaceutically active ingredient capable of oral inhalation to drive off the pharmaceutical solvent; and
(d) cutting, dividing or perforating the inert supporting material on which is deposited or in which is impregnated the pharmaceutically active ingredient capable of oral inhalation to obtain a dosage unit holding a defined therapeutically effective amount of the pharmaceutically active ingredient capable of oral inhalation.

23. The method of preparing a pharmaceutical composition in unit dosage form defined in claim 20 wherein prior to step (b) the inert supporting material is cut, divided or perforated to obtain a pre-shaped and pre-sized inert supporting material of a size capable of holding a dosage unit of the pharmaceutically active ingredient capable of oral inhalation, the formulation concentrate containing the pharmaceutically active ingredient is then applied to the pre-sized inert supporting material to deposit said pharmaceutically active ingredient on or impregnate said pharmaceutically active ingredient in said supporting material, and then said supporting material is dried to obtain the dosage unit holding a defined therapeutically effective amount of the pharmaceutically active ingredient capable of oral inhalation.

24. The method of preparing a pharmaceutical composition in unit dosage form defined in claim 20 wherein according to step (b) the formulation concentrate is applied to the inert supporting material by means of spraying, dispensing, pipetting, inkjet printing, or dipping the inert supporting material into a bath of formulation concentrate.

25. The pharmaceutical composition in unit dosage form defined in claim 4 wherein the pharmaceutically active ingredient is selected from the group consisting of asthma drugs, respiratory disorder drugs, and antibiotics.

26. A method for nasal or oral respiratory route administration of a pharmaceutically active ingredient capable of nasal or oral inhalation as a nebulizing mist to a patient which comprises the steps of:
(a) contacting an inert supporting material, which when wet maintains its integrity, carrying a unit dosage quantity of an aerosol-administrable pharmaceutically active ingredient with a reconstituting solvent for said pharmaceutically active ingredient whereby said pharmaceutically active ingredient is leached from said support into said reconstituting solvent to form a reconstituting solution containing said pharmaceutically active ingredient;
(b) dispersing said reconstituting solution into an aerosol; and
(c) delivering said aerosol to a respiratory tract of a patient requiring said pharmaceutically active ingredient.
